(19) United States
(12) Patent Application Publication
(10) Pub. No.: US 2024/0366542 A1
(43) Pub. Date: Nov. 7, 2024

(54) SULFOXIMINE ACTIVATORS OF SEROTONIN RECEPTORS

(71) Applicant: ATAI Therapeutics, Inc., New York, NY (US)

(72) Inventors: Tanweer A. Khan, Encinitas, CA (US); Alan C. Gibbs, Encinitas, CA (US); Glenn F. Short, Encinitas, CA (US); Robert B. Perni, Encinitas, CA (US)

(21) Appl. No.: 18/532,940

(22) Filed: Dec. 7, 2023

Related U.S. Application Data
(60) Provisional application No. 63/430,989, filed on Dec. 7, 2022.

(51) Int. Cl. A61K 31/18; C07C 313/06

(52) U.S. Cl. CPC A61K 31/18; C07C 313/06

(57) ABSTRACT

Methods of treating a stress-related disease or disorder such as PTSD and mood/depressive disorder, inducing neurite outgrowth, or inducing structural neuroplasticity, comprising administering to a subject a composition comprising an effective amount of a compound that does not have an agonist activity on 5-HT_{2A} serotonin receptor are disclosed. Also disclosed are pharmaceutical compositions comprising compound not having an agonist activity on 5-HT_{2A} serotonin receptor and a pharmaceutically acceptable carrier.

### CROSS-REFERENCE TO RELATED APPLICATIONS

This application claims benefit of priority under 35 U.S.C. § 119 (e) of U.S. Provisional Application No. 63/430,989, filed Dec. 7, 2022. The disclosure of the prior application is incorporated herein by reference in its entirety.

### BACKGROUND OF THE INVENTION

### Field of the Invention

This application relates to the field of treating stress related disorders and specifically to novel compounds and the use of such compounds in treating stress related disorders.

### Background

Rapid and effective treatments for stress-related disorders, such as depression, anxiety, and post-traumatic stress disorder, remain a significant unmet medical need. Studies in humans and animals have shown that depression and stress exposure induce structural and functional changes in the brain. Signs of neuronal atrophy have been found in brain regions involved in stress-related behaviors, including prefrontal cortex and hippocampus. In animals, these structural changes have been shown to include loss of neurites, dendritic spines and synaptic contacts, as well as reduced hippocampal neurogenesis. Studies in humans and animals also have shown that depression and stress exposure decrease cerebral cortex and hippocampal levels of brain-derived neurotrophic factor (BDNF), which promotes neuronal survival and synaptic plasticity. Together, these findings are consistent with the hypothesis that reduced BDNF may play a role in stress-related neuronal structural changes. In support of this hypothesis, chronic but not acute administration of typical antidepressant drugs, such as selective serotonin (5-HT) reuptake inhibitors (SSRIs), attenuate the effects of stress on neurogenesis, neuronal structure and BDNF levels in animals and demonstrate antidepressant and anxiolytic effects in humans. In addition, single administration of ketamine attenuates these effects of stress in animals and demonstrates rapid-acting antidepressant effects in humans. Thus, compounds that promote the generation and/or maintenance of neurites, spines, synapses and/or neurons upon single administration may have rapid therapeutic benefit in the treatment of stress-related disorders.

Tryptamines are a structural class of compounds containing an indolealkylamine backbone with reported psychedelic and non-psychedelic effects. Psychedelic tryptamines include compounds such as psilocybin, its active metabolite psilocin, and N,N-dimethyltryptamine (DMT). Psilocybin and psilocin are found in hundreds of species of hallucinogenic mushrooms and were used in Aztec rituals. DMT is present in ayahuasca, a hallucinogenic brew traditionally used in ceremonial settings in South America. Psilocybin and ayahuasca have been reported to induce rapid and lasting clinical antidepressant efficacy following single administration. Data in rodents and nonhuman primates also suggest the potential for antidepressant-like effects of psilocybin, DMT or ayahuasca. These compounds exert functional activity at a variety of central nervous system receptors, including serotonin receptors, of which partial agonism at 5-HT_{2A} receptors is believed to mediate the psychedelic effects. Psychedelic tryptamines have been reported to exhibit neuronal plasticity-promoting effects, including neuritogenesis, spinogenesis, synaptogenesis, hippocampal cell proliferation and/or hippocampal neurogenesis, which may underlie their therapeutic benefit in the treatment of stress-related disorders, such as depression. Specifically, psilocin and DMT were reported to significantly increase measures of neurite outgrowth in primary embryonic rat cortical neurons. There is published data on the effects of the reference 5-HT_{2A} agonist, DOI, and antagonist, M100907, in NGF-induced neurite outgrowth in rat adrenal medulla pheochromocytoma-derived PC12 cells.

There is a need for novel compositions for the treatment of stress related disorders. Given the reported 5-HT_{2A} receptor agonist activity and ability to promote neurite outgrowth associated with classical psychedelic compounds novel compounds may provide an improved therapeutic index for treating stress related disorders with reduced side effect profiles.

### SUMMARY OF THE INVENTION

The present invention exploits the different pharmacological and toxicological profiles, and thus the different functional characteristics possessed by novel compound having an agonist activity on 5-HT_{2A} serotonin receptor. Specifically, those novel compounds induce neurite growth increasing neural plasticity and have many of the functional properties desirable for treating stress-related diseases or disorders such as PTSD and mood/depressive disorder.

Some embodiments are directed towards a method of treating a stress-related disease or disorder in a subject in need thereof, comprising administering to the subject a therapeutically effective amount of a compound of Formula I, a derivative thereof, or a combination thereof.

Some embodiments are directed towards a method of inducing neurite outgrowth in a subject in need thereof, comprising administering to the subject a therapeutically effective amount of a compound of Formula I, a derivative thereof, or a combination thereof.

Some embodiments are directed towards a method of treating neuronal atrophy in a subject in need thereof, comprising administering to the subject a therapeutically effective amount of a compound of Formula I, a derivative thereof, or a combination thereof.

Some embodiments are directed towards a method of inducing structural neuroplasticity in a subject in need thereof, comprising administering to the subject a therapeutically effective amount of a compound of Formula I, a derivative thereof, or a combination thereof.

In an embodiment, the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), or any combination thereof. In an embodiment, the stress-related disease or disorder is depression, anxiety or post-traumatic stress disorder.

In an embodiment, the compound has antidepressant and anxiolytic effects.

In an embodiment, the administering includes intracutaneous, subcutaneous, intravenous, intraarterial, intradermal, transdermal, oral, sublingual buccal, or nasal route of administration. In an embodiment, the methods include administering the compound as a single dose. In an embodiment, the methods include administering the compound in repeated doses.

In an embodiment, administration of the compound induces neurite outgrowth. In an embodiment neurite outgrowth includes neurite number, neurite total length, number of neurite branch points per neuron or any combination thereof. In an embodiment, the neurite outgrowth includes neurite outgrowth on prefrontal cortex neurons and/or hippocampal neurons.

Some embodiments are directed to the administration of the compound of Formula I and a therapeutic agent for the treatment of a stress related disorder, treating neural atrophy, inducing neural plasticity and/or inducing neurite growth. In an embodiment, the therapeutic agent is administered simultaneously with, prior to or following administration of the compound of Formula I.

Some embodiments are directed to pharmaceutical compositions having the compound of Formula I and a pharmaceutically acceptable carrier. In an embodiment, the pharmaceutically acceptable carrier is saline or purified water.

### BRIEF DESCRIPTION OF THE DRAWINGS

FIG. 1 shows a graph of 5-HTR2A functional activity for compound 3.

FIG. 2 shows a graph of 5-HTR2B functional activity for compound 3.

FIG. 3 shows a graph of 5-HTR2A beta-arrestin functional activity for compound 3.

FIG. 4 shows a graph of 5-HTR2C functional activity for compound 3.

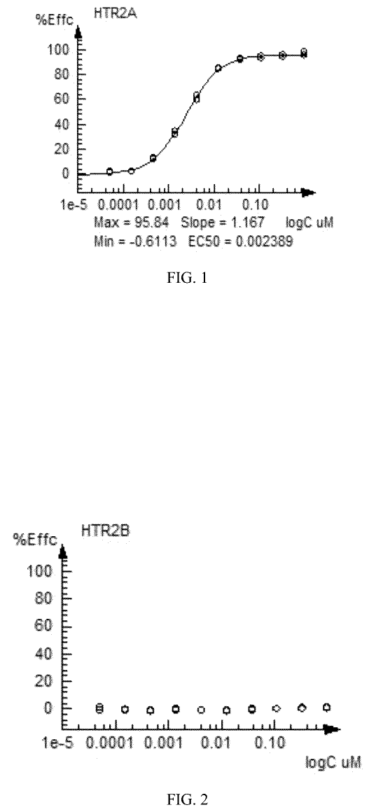

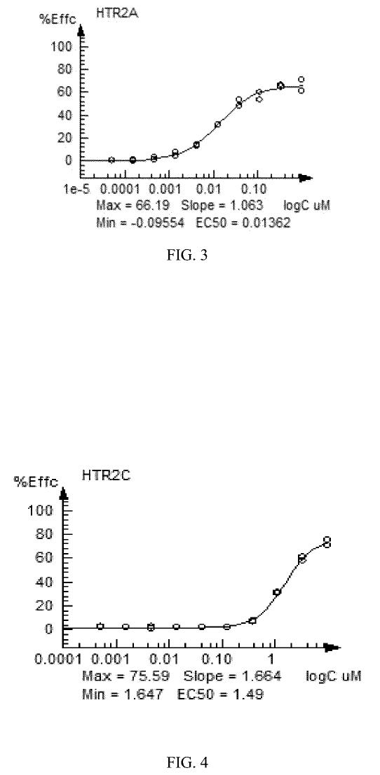

### DETAILED DESCRIPTION OF THE INVENTION

The present invention exploits the different pharmacological and toxicological profiles, and thus the different functional characteristics possessed by novel compounds that do not have an agonist activity on 5-HT_{2A} serotonin receptor. Specifically, those novel compounds induce neurite growth increasing neural plasticity and have many of the functional properties desirable for treating stress-related diseases or disorders such as PTSD and mood/depressive disorder.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by a person skilled in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50 mg means in the range of 45 mg to 55 mg.

The terms “administration of” and or “administering” should be understood to mean providing a composition in a therapeutically effective amount to the subject in need of treatment. The compositions may be administered by any route, taking into consideration the specific condition for which it has been selected. The compositions may be delivered orally, by injection, inhalation (including orally, intranasally, and intratracheally), ocularly, transdermally (via simple passive diffusion formulations or via facilitated delivery using, for example, iontophoresis, microporation with microneedles, radio-frequency ablation, or the like), intravascularly, cutaneously, subcutaneously, intramuscularly, sublingually, intracranially, epidurally, rectally, intravesically, and vaginally, among others.

As used herein, the term “a derivative thereof” refers to a salt thereof, a pharmaceutically acceptable salt thereof, an ester thereof, a free acid form thereof, a free base form thereof, a solvate thereof, a co-crystal thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination thereof.

As used herein “neuroplasticity” refers to any neuronal plasticity, which can include for example neurite outgrowth. Neurite outgrowth can broadly refer to various parameters than can be measured on neurites, including but not limited to neurite number, neurite total length, number of neurite branch point per neuron or any combination thereof. Neuronal plasticity in general and neurite outgrowth in particular can occur in any neurons and neurites in the brain, including but not limited to prefrontal cortex neurons and/or hippocampal neurons. As used herein, a “disease or disorder that can benefit from neuroplasticity” may include any disease or disorder that can be treated by, or than can see one or more of its symptoms alleviate by a change in neuroplasticity in the patient's brain, for example by neurite outgrowth, such as neurite outgrowth in prefrontal cortex neurons and/or hippocampal neurons.

As used herein, “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. Examples of active ingredient include, but are not limited to, chemical compound, drug, therapeutic agent, small molecule, etc. In an embodiment, the active ingredient is a compound of Formula I, a derivative thereof, or a combination thereof.

As used herein, “pharmaceutically acceptable” it is meant the carrier, diluent or excipient must be compatible with the other ingredients of the formulation and not deleterious to the recipient thereof, nor to the activity of the active ingredient of the formulation.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total, whether induction of or maintenance of), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease and prolonging disease-free survival as compared to disease-free survival if not receiving treatment and prolonging disease-free survival as compared to disease-free survival if not receiving treatment.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of a stress-related disease or disorder). The effective amount can be determined as described herein.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein.

Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term, “compound,” as used herein is meant to include all stereoisomers, geometric isomers, and tautomers of the structures depicted.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “halo,” or “halogen,” as used herein, alone or in combination, refers to fluorine, chlorine, bromine, or iodine.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

### Compounds

Some embodiments are directed towards a compound, or a derivative thereof, of Formula I:

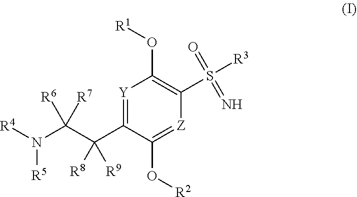

wherein:
   R¹ and R² are independently H, D, C₁-C₆ alkyl, or C₁-C₆ fluoroalkyl;
   R³ is H, C₁-C₆ alkyl, C₁-C₆ fluoroalkyl, CH₂X, CHX₂, or CX₃ wherein the alkyl or fluoroalkyl is optionally substituted with one or more D;
   R⁴ is H, C₁-C₆ alkyl, or C₁-C₆ fluoroalkyl wherein the alkyl or fluoroalkyl is optionally substituted with a phenyl or a C₁-C₅ heteroaryl and wherein the phenyl or heteroaryl is optionally substituted with one or more of D, a halogen, or a C₁-C₆ alkyl;
   R⁵ is H, a C₁-C₆ alkyl, or a C₁-C₆ fluoroalkyl;
   R⁶, R⁷, R⁸, and R⁹ are independently H, D, a C₁-C₆ alkyl, a C₁-C₆ fluoroalkyl, a halogen, a C₁-C₇ heterocycloalkyl, a C₃-C₈ cycloalkyl, a C₁-C₇ heteroaryl, —OH, or —OD; or R⁶ and R⁷ or R⁸ and R⁹ are independently taken together to form ═O;
   X is at each instance is independently F, Cl, Br, or I;
   Y is CR¹⁰ or N;
   Z is CR¹¹ or N;
   R¹⁰ and R¹¹ are independently H, D, CN, a halogen, OH, OD, OCH₃, CF₃, or COOR¹²; and
   R¹² is H, a C₁-C₆ alkyl, a C₁-C₆ fluoroalkyl, or a C₁-C₇ heterocycloalkyl.

In some embodiments, the compound of Formula I, or a derivative thereof, may be selected from:

| Compound | | |
|---|---|---|
| # | Structure | Name |
| 1 | 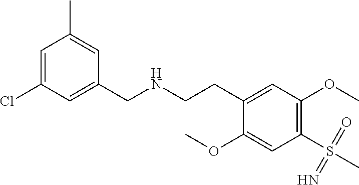 [SMILES: COc1cc(S(C)(=N)=O)c(OC)cc1CCNCc1cc(C)cc(Cl)c1] | (4-(2-((3-chloro-5-methylbenzyl)amino)ethyl)-2,5- dimethoxyphenyl)(imino)(methyl)-λ⁶-sulfanone |
| 2 | 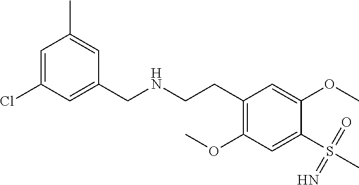 [SMILES: COc1cc(S(C)(=N)=O)c(OC)cc1CCNCc1cc(C)cc(Cl)c1] | (4-(2-((3-chloro-5-methylbenzyl)amino)ethyl)-2,5- dimethoxyphenyl)(imino)(methyl)-λ⁶-sulfanone Enantiomer A |
| 3 | 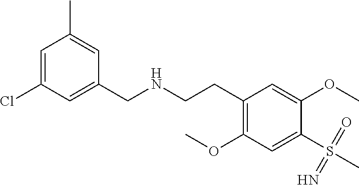 [SMILES: COc1cc(S(C)(=N)=O)c(OC)cc1CCNCc1cc(C)cc(Cl)c1] | (4-(2-((3-chloro-5-methylbenzyl)amino)ethyl)-2,5- dimethoxyphenyl)(imino)(methyl)-λ⁶-sulfanone Enantiomer B |
| 4 | 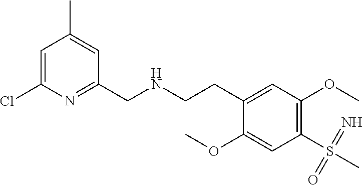 [SMILES: COc1cc(S(C)(=N)=O)c(OC)cc1CCNCc1cc(C)cc(Cl)n1] | (4-(2-(((6-chloro-4-methylpyridin-2- yl)methyl)amino)ethyl)-2,5- dimethoxyphenyl)(imino)(methyl)-λ⁶-sulfanone |
| 5 | 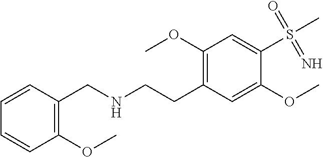 [SMILES: COc1cc(S(C)(=N)=O)c(OC)cc1CCNCc1ccccc1OC] | (2,5-dimethoxy-4-(2-((2- methoxybenzyl)amino)ethyl)phenyl)(imino) (methyl)-λ⁶-sulfanone |
| 6 | 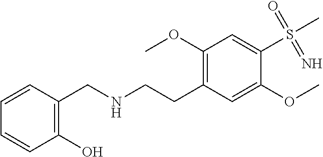 [SMILES: COc1cc(S(C)(=N)=O)c(OC)cc1CCNCc1ccccc1O] | (4-(2-((2-hydroxybenzyl)amino)ethyl)-2,5- dimethoxyphenyl)(imino)(methyl)-λ⁶-sulfanone |
| 7 | 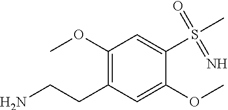 [SMILES: COc1cc(S(C)(=N)=O)c(OC)cc1CCN] | (4-(2-aminoethyl)-2,5- dimethoxyphenyl)(imino)(methyl)-λ⁶-sulfanone |
| 8 | 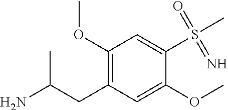 [SMILES: COc1cc(S(C)(=N)=O)c(OC)cc1CC(C)N] | (4-(2-aminopropyl)-2,5- dimethoxyphenyl)(imino)(methyl)-λ⁶-sulfanone |
| 9 | 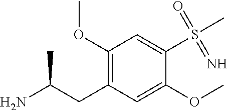 [SMILES: COc1cc(S(C)(=N)=O)c(OC)cc1C[C@H](C)N] | (4-((S)-2-aminopropyl)-2,5- dimethoxyphenyl)(imino)(methyl)-λ⁶-sulfanone |
| 10 | 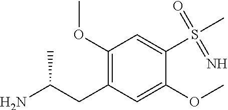 [SMILES: COc1cc(S(C)(=N)=O)c(OC)cc1C[C@@H](C)N] | (4-((R)-2-aminopropyl)-2,5- dimethoxyphenyl)(imino)(methyl)-λ⁶-sulfanone |
| 11 | 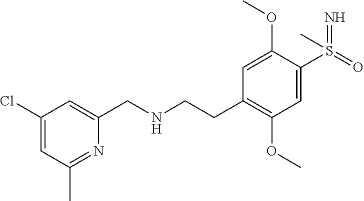 [SMILES: COc1cc(S(C)(=N)=O)c(OC)cc1CCNCc1cc(Cl)cc(C)n1] | (4-(2-(((4-chloro-6-methylpyridin-2- yl)methyl)amino)ethyl)-2,5- dimethoxyphenyl)(imino)(methyl)-λ⁶-sulfanone |
| 12 | 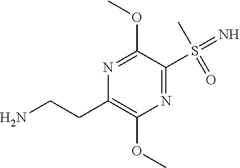 [SMILES: COc1nc(S(C)(=N)=O)c(OC)nc1CCN] | (5-(2-aminoethyl)-3,6-dimethoxypyrazin-2- yl)(imino)(methyl)-λ⁶-sulfanone |
| 13 | 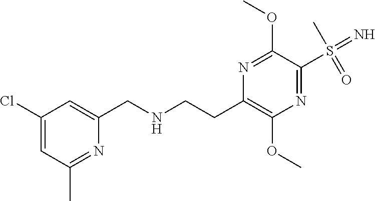 [SMILES: COc1nc(S(C)(=N)=O)c(OC)nc1CCNCc1cc(Cl)cc(C)n1] | (5-(2-(((4-chloro-6-methylpyridin-2- yl)methyl)amino)ethyl)-3,6-dimethoxypyrazin-2- yl)(imino)(methyl)-λ⁶-sulfanone |
| 14 | 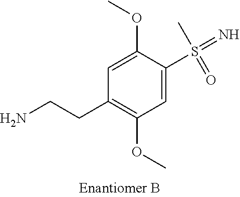 [SMILES: COc1cc(S(C)(=N)=O)c(OC)cc1CCN] | (4-(2-aminoethyl)-2,5- dimethoxyphenyl)(imino)(methyl)-λ⁶-sulfanone |
| 15 | 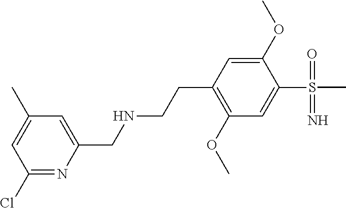 [SMILES: COc1cc(S(C)(=N)=O)c(OC)cc1CCNCc1cc(C)cc(Cl)n1] | (4-(2-(((6-chloro-4-methylpyridin-2- yl)methyl)amino)ethyl)-2,5- dimethoxyphenyl)(imino)(methyl)-λ⁶-sulfanone |
| 16 | 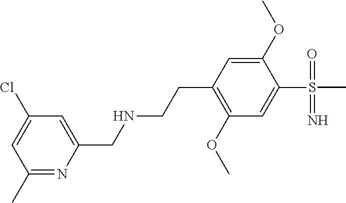 [SMILES: COc1cc(S(C)(=N)=O)c(OC)cc1CCNCc1cc(Cl)cc(C)n1] | (4-(2-(((4-chloro-6-methylpyridin-2- yl)methyl)amino)ethyl)-2,5- dimethoxyphenyl)(imino)(methyl)-λ⁶-sulfanone |
| 17 | 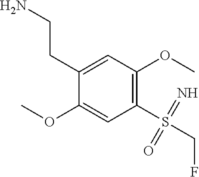 [SMILES: COc1cc(S(=N)(=O)CF)c(OC)cc1CCN] | (4-(2-aminoethyl)-2,5- dimethoxyphenyl)(fluoromethyl)(imino)-1⁶- sulfanone |
| 18 | 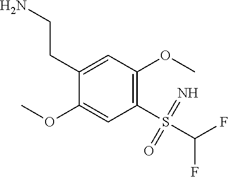 [SMILES: COc1cc(S(=N)(=O)C(F)F)c(OC)cc1CCN] | (4-(2-aminoethyl)-2,5- dimethoxyphenyl)(difluoromethyl)(imino)-1⁶- sulfanone |
| 19 | 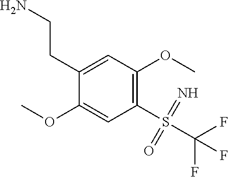 [SMILES: COc1cc(S(=N)(=O)C(F)(F)F)c(OC)cc1CCN] | (4-(2-aminoethyl)-2,5- dimethoxyphenyl)(imino)(trifluoromethyl)-1⁶- sulfanone |
| 20 | 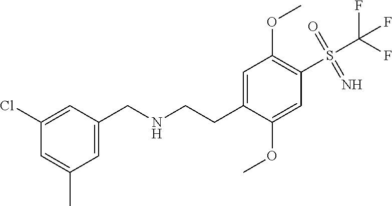 [SMILES: COc1cc(S(=N)(=O)C(F)(F)F)c(OC)cc1CCNCc1cc(C)cc(Cl)c1] | (4-(2-((3-chloro-5-methylbenzyl)amino)ethyl)-2,5- dimethoxyphenyl)(imino)(trifluoromethyl)-1⁶- sulfanone |
| 21 | 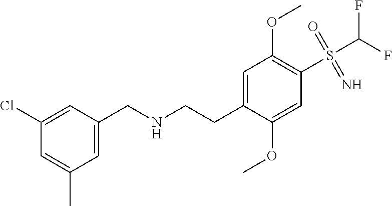 [SMILES: COc1cc(S(=N)(=O)C(F)F)c(OC)cc1CCNCc1cc(C)cc(Cl)c1] | (4-(2-((3-chloro-5-methylbenzyl)amino)ethyl)-2,5- dimethoxyphenyl)(difluoromethyl)(imino)-1⁶- sulfanone |
| 22 | 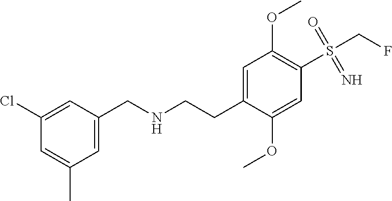 [SMILES: COc1cc(S(=N)(=O)CF)c(OC)cc1CCNCc1cc(C)cc(Cl)c1] | (4-(2-((3-chloro-5-methylbenzyl)amino)ethyl)-2,5- dimethoxyphenyl)(fluoromethyl)(imino)-1⁶- sulfanone |
| 23 | 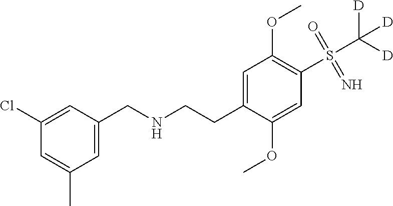 [SMILES: [2H]C([2H])([2H])S(=N)(=O)c1cc(OC)c(CCNCc2cc(C)cc(Cl)c2)cc1OC] | (4-(2-((3-chloro-5-methylbenzyl)amino)ethyl)-2,5- dimethoxyphenyl)(imino)(methyl-d₃)-1⁶-sulfanone |

or a derivative thereof.

### Pharmaceutical Compositions

Embodiments herein are directed to a pharmaceutical composition comprising a compound of Formula I, a derivative thereof, or a combination thereof, and a pharmaceutically acceptable excipient.

While it may be possible for the compounds described herein to be administered as the raw chemical, it is also possible to present them as a pharmaceutical composition. Accordingly, provided herein are pharmaceutical compositions which comprise one or more of certain compounds disclosed herein, or a derivative thereof, together with one or more pharmaceutically acceptable excipients thereof and optionally one or more other therapeutic ingredients. The excipient(s) must be “acceptable” in the sense of being compatible with the other ingredients of the formulation and not deleterious to the recipient thereof. Proper formulation of the pharmaceutical composition is dependent upon the route of administration chosen. Any of the well-known techniques and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

In some embodiments, the pharmaceutical compositions for use in accordance with embodiments herein can be formulated in conventional manner using one or more physiologically acceptable excipients.

When employed as pharmaceuticals, the compounds can be administered in the form of pharmaceutical compositions. These compositions can be prepared in a manner well known in the pharmaceutical arts, and can be administered by a variety of routes, depending upon whether local or systemic treatment is desired and upon the area to be treated.

Administration of the disclosed compounds or compositions may be oral administration. Conventional pharmaceutical carriers, aqueous, powder or oily bases, thickeners and the like may be necessary or desirable. The compounds can be contained in such formulation's pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance. For example, Modern Pharmaceutics, 5th Edition, Banker & Rhodes, CRC Press (2009); and Goodman & Gilman's The Pharmaceutical Basis of Therapeutics, 13th Edition, McGraw Hill, New York (2018) can be consulted.

In some embodiments, a method of treating a disease or disorder associated with sodium channel mediated activity comprises administering a compound or a pharmaceutical composition of embodiments disclosed herein. In some embodiments, the compound is in a therapeutically effective amount. In some embodiments, the therapeutically effective amount is an amount disclosed herein.

Some embodiments disclosed herein also include pharmaceutical compositions which contain, as the active ingredient, one or more of the compounds disclosed herein in combination with one or more pharmaceutically acceptable carriers (excipients).

In some embodiments, a method of making a pharmaceutical composition comprises mixing the active ingredient with an excipient, diluting the active ingredient using an excipient, or enclosing the active ingredient within a carrier in the form of, for example, a capsule, sachet, paper, or other container. When the excipient serves as a diluent, it can be a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, as well as soft and hard gelatin capsules.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The pharmaceutical compositions can additionally include lubricating agents such as talc, magnesium stearate, and mineral oil; wetting agents; emulsifying and suspending agents; preserving agents such as methyl- and propylhydroxy-benzoates; sweetening agents; and flavoring agents. The pharmaceutical compositions can be formulated so as to provide quick, sustained or delayed release of the active ingredient after administration to the patient by employing procedures known in the art.

The pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. The compositions include those suitable for oral, parenteral (including subcutaneous, intradermal, intramuscular, intravenous, intraarticular, and intramedullary), intraperitoneal, intrathecal, intradural, transmucosal, transdermal, rectal, intranasal, topical (including, for example, dermal, buccal, sublingual and intraocular), intravitreal, or intravaginal administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound disclosed herein or a derivative thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

Compositions of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All compositions for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For preparing solid compositions such as tablets, the principal active ingredient can be mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the pharmaceutical composition so that the pharmaceutical composition can be readily subdivided into equally therapeutically effective unit dosage forms such as tablets, pills and capsules. This solid pre-formulation is then subdivided into unit dosage forms of the type described above containing from, for example, about 0.01 to about 1000 mg of the active ingredient.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

The compounds may be formulated for parenteral administration by injection, e.g., by bolus injection or continuous infusion. Compositions for injection may be presented in unit dosage form, e.g., in ampoules or in multi-dose containers, with an added preservative. The pharmaceutical compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

In some embodiments, the pharmaceutical compositions administered to a patient can be in the form of pharmaceutical compositions described above. In some embodiments, these compositions can be sterilized by conventional sterilization techniques, or may be sterile filtered. Aqueous solutions can be packaged for use as is, or lyophilized, the lyophilized preparation being combined with a sterile aqueous carrier prior to administration. In some embodiments, the pH of the compound preparations is about 3 to about 11, about 5 to about 9, about 5.5 to about 6.5, or about 5.5 to about 7.5. It will be understood that use of certain of the foregoing excipients, carriers, or stabilizers will result in the formation of pharmaceutical salts.

Preferred unit dosage pharmaceutical compositions are those containing an effective dose, as herein below recited, or an appropriate fraction thereof, of the active ingredient.

It should be understood that in addition to the ingredients particularly mentioned above, the pharmaceutical compositions described above may include other agents conventional in the art having regard to the type of pharmaceutical composition in question, for example those suitable for oral administration may include flavoring agents.

In some embodiments, the therapeutically effective amount can vary according to, for example, the particular use for which the treatment is made, the manner of administration of the compound, the health and condition of the patient, and the judgment of the prescribing physician. The proportion or concentration of a compound in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, composition of the excipient, and its route of administration. Effective doses can be extrapolated from dose-response curves derived from in vitro or animal model test systems.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

The active compound can be effective over a wide dosage range and can be generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The amount of active ingredient that may be combined with the carrier materials to produce a single dosage form will vary depending upon the host treated and the particular mode of administration.

The precise amount of compound administered to a patient will be the responsibility of the attendant physician. The specific dose level for any particular patient will depend upon a variety of factors including the activity of the specific compound employed, the age, body weight, general health, sex, diet, time of administration, route of administration, rate of excretion, drug combination, the precise disorder being treated, and the severity of the indication or condition being treated. In addition, the route of administration may vary depending on the condition and its severity.

In an embodiment, the pharmaceutically acceptable carrier is saline or purified water.

The pharmaceutical composition can be used in any of the methods disclosed herein.

The pharmaceutical compositions described herein can be formulated, for example, by employing conventional vehicles or diluents, as well as additives of a type appropriate to the mode of desired administration (for example, excipients, preservatives, etc.) according to techniques known in the art of pharmaceutical formulation. The pharmaceutical compositions described herein can also be formulated as is, without any carrier. The pharmaceutical compositions can be formulated in a variety of unit dosage forms depending upon the method of administration. Suitable unit dosage forms, include, but are not limited to powders, tablets, pills, capsules, lozenges, sprays, granules, etc.

### Methods of Treating Stress-Related Diseases and Disorders

Some embodiments are directed towards a method of treating a stress-related disease or disorder in a subject in need thereof, comprising administering to the subject a therapeutically effective amount of a compound of Formula I, a derivative thereof, or a combination thereof.

The dosage of the composition to achieve a therapeutic effect will depend on factors such as the formulation, pharmacological potency of the composition, age, weight and sex of the patient, condition being treated, severity of the patient's symptoms, route of delivery, and response pattern of the patient. It is also contemplated that the treatment and dosage of the compositions may be administered in unit dosage form and that one skilled in the art would adjust the unit dosage form accordingly to reflect the relative level of activity. The decision as to the particular dosage to be employed (and the number of times to be administered per day) is within the competency and discretion of a skilled physician and may be varied by titration of the dosage to the particular circumstances to produce the therapeutic effect. Further, one of skill in the art would be able to calculate any changes in effective amounts of the compositions due to changes in the composition components or dilutions. In one aspect, the compositions may be diluted 2-fold. In another aspect, the compositions may be diluted 4-fold. In a further aspect, the compositions may be diluted 8-fold.

In an embodiment, the methods include administering the compound as a single dose. In an embodiment, the methods include administering the compound in repeated doses.

The effective amounts may be provided as a single dose or on regular schedule, i.e., on a daily, weekly, monthly, or yearly basis or on an irregular schedule with varying administration days, weeks, months, etc. To reduce the occurrence of possible side effect associated with the dose, an effective amount may be provided as a split dose, where the single dose is split into two doses, that are administered apart, usually over several hours. For example, a single dose may be split into two doses, administered 1 hour apart, 2 hours apart, 3 hours apart, 4 hours apart, 5 hours apart, 6 hours apart, 7 hours apart, 8 hours apart, or more. Alternatively, the therapeutically effective amount to be administered may vary. In one aspect, the therapeutically effective amount for the first dose is higher than the therapeutically effective amount for one or more of the subsequent doses. In another aspect, the therapeutically effective amount for the first dose is lower than the therapeutically effective amount for one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In an embodiment, the administering includes intracutaneous, subcutaneous, intravenous, intraarterial, intradermal, transdermal, oral, sublingual, buccal, or nasal route of administration.

Although the compositions may be administered alone, they may also be administered in the presence of one or more pharmaceutical carriers that are physiologically compatible. The carriers may be in dry or liquid form and must be pharmaceutically acceptable. Liquid compositions may be sterile solutions or suspensions. When liquid carriers are utilized, they may be sterile liquids. Liquid carriers may be utilized in preparing solutions, suspensions, emulsions, syrups and elixirs. In one aspect, the compositions may be dissolved a liquid carrier. In another aspect, the compositions may be suspended in a liquid carrier. One of skill in the art of formulations would be able to select a suitable liquid carrier, depending on the route of administration. The compositions may alternatively be formulated in a solid carrier. In one aspect, the composition may be compacted into a unit dose form, i.e., tablet or caplet. In another aspect, the composition may be added to unit dose form, i.e., a capsule. In a further aspect, the composition may be formulated for administration as a powder. The solid carrier may perform a variety of functions, i.e., may perform the functions of two or more of the excipients described below. For example, a solid carrier may also act as a flavoring agent, lubricant, solubilizer, suspending agent, filler, glidant, compression aid, binder, disintegrant, or encapsulating material. In one aspect, a solid carrier acts as a lubricant, solubilizer, suspending agent, binder, disintegrant, or encapsulating material. The composition may also be sub-divided to contain appropriate quantities of the compositions. For example, the unit dosage can be packaged compositions, e.g., packeted powders, vials, ampoules, prefilled syringes or sachets containing liquids.

Where the compositions include a pharmaceutical carrier(s), the amount of the pharmaceutical carrier(s) is determined by the solubility and chemical nature of the peptides, chosen route of administration and standard pharmacological practice. The pharmaceutical carrier(s) may be solid or liquid and may incorporate both solid and liquid carriers/matrices. A variety of suitable liquid carriers is known and may be readily selected by one of skill in the art. Such carriers may include, e.g., dimethylsulfoxide (DMSO), saline, buffered saline, purified water, cyclodextrin, hydroxypropyl-beta-cyclodextrin (HPBCD), n-dodecyl-β-D-maltoside (DDM) and mixtures thereof. Similarly, a variety of solid (rigid or flexible) carriers and excipients are known to those of skill in the art.

In an embodiment, the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

PTSD is a serious, chronic, life-threatening psychiatric disorder. Psychiatric symptoms of PTSD are debilitating and occur after experiencing a single traumatic event or repeated traumatic experiences, such as violence, accidents, sexual and/or childhood abuse, natural disasters, terrorism, and war. Symptoms include recurring and intrusive negative thoughts or recollections of the traumatic event, cognitive disruption, hyperarousal to event related cues, and avoidance behaviors that persist for longer periods than a month after experiencing a traumatic event. Overall reduction in the quality of life is common in individuals with PTSD leading to disability and can affect physical health with manifestation of other comorbidities such as cardiovascular disease, concomitant mental health conditions and suicidality.

Two serotonin reuptake inhibitors (SSRIs) are currently approved for the treatment of PTSD, sertraline (Zoloft®) and paroxetine (Paxil®), which, increase the level of serotonin in the synaptic cleft. Sertraline and paroxetine have demonstrated moderate efficacy in reducing PTSD symptoms, but rarely result in full disorder remission. These pharmacotherapies also have problematic side effects and generally require long-term and/or consistent use to maintain effectiveness, although long-term compliance is poor. Studies have demonstrated that administering stand-alone sertraline or paroxetine reported significantly decreased reduction in PTSD symptoms compared to currently available behavioral interventions.

Based on the low response rate to existing pharmacotherapy, the most recent clinical practice guidelines recommend psychotherapy as the first-line treatment for PTSD. Specifically, the American Psychological Association (APA) and US Departments of Defense and Departments of Veterans Affairs (DoD/VA) practice guidelines recommend Cognitive Processing Therapy (CPT), Cognitive Behavioral Therapy (CBT), Prolonged Exposure Therapy (PET), Brief Eclectic Psychotherapy (BEP), Narrative Exposure Therapy (NAT), and Eye-Movement Desensitization and Reprocessing (EMDR), as first-line treatment for PTSD as these treatments have repeatedly demonstrated efficacy in reducing symptoms of PTSD in randomized clinical trials. However, it has been shown that CPT and PE therapy did not lead to remission or even clinically meaningful reductions in symptoms in the majority of patients with PTSD. This is consistent with several studies that estimate that between 40%-60% of patients receiving any treatment for PTSD do not respond adequately and/or continue to meet diagnostic criteria after receiving treatment.

One of the symptoms of PTSD is learned avoidance behavior. Avoidance is a safety-seeking or protective response in response to trauma. However, as this avoidance behavior becomes more extreme, a person's quality of life may lessen. One type of behavioral therapy for PTSD is Exposure Therapy which can reduce anxiety and ultimately eliminating avoidance behavior and improving the quality of life for a subject having PTSD.

Depressive disorders are characterized by low mood, feeling sad or hopeless, increased irritability, sleep disturbance, lowered energy and feeling tired, poor concentration, lowered self-esteem or feeling worthless, lack of interest or pleasure in things, slowed movement, thinking they would be better off dead and self-harm, and actively considering and attempting suicide, and include major depressive disorder, bipolar depression, treatment resistant depression, and dysthymic disorder.

In an embodiment, the stress-related disease or disorder is depression, anxiety or post-traumatic stress disorder.

In an embodiment, the compound has antidepressant and anxiolytic effects.

Serotonin receptors (i.e., 5-HT receptors), are a group of G protein-coupled receptors and ligand-gated ion channels found in the central and peripheral nervous systems. The receptors mediate both excitatory and inhibitory neurotransmission. The 5-HT receptors modulate the release of many neurotransmitters, including glutamate, GABA, dopamine, epinephrine/norepinephrine, and acetylcholine, as well as many hormones, including oxytocin, prolactin, vasopressin, cortisol, corticotropin, and substance P, among others. The 5-HT receptors influence various biological and neurological processes such as aggression, anxiety, appetite, cognition, learning, memory, mood, nausea, sleep, and thermoregulation. They are the target of a variety of pharmaceutical and recreational drugs, including many antidepressants, antipsychotics, anorectics, antiemetics, gastroprokinetic agents, antimigraine agents, hallucinogens, and entactogens. There are a variety of 5-HT receptors (e.g., 5-HT₁, 5-HT₂, 5-HT₃, 5-HT₄, 5-HT₅, 5-HT₆, 5-HT₇) which have differing functions. There are also 5-HT receptor subtypes (e.g., 5-HT_{1A}, 5-HT_{1B}, 5-HT_{1D}, 5-HT_{1E}, 5-HT_{1F}, 5-HT_{2A}, 5-HT_{2B}, 5-HT_{2C}, 5-HT_{5A}, 5-HT_{5B}). For example, 5-HT_{2A} is involved in addiction, anxiety, appetite, cognition, imagination, learning, memory, mood, perception, sexual behavior, sleep, thermoregulation and vasoconstriction. In another example, 5-HT_{2C} is involved in addiction, anxiety, appetite, GI motility, heteroreceptor for norepinephrine and dopamine, locomotion, mood, sexual behavior, sleep, thermoregulation and vasoconstriction.

The compositions comprising a compound of Formula I, a derivative thereof, or a combination thereof, can be administered alone or in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to stress-related diseases or disorders. In an embodiment, the therapeutic agent is administered simultaneously with, prior to or following administration of the compound of Formula I.

Therapeutic agents for combination treatments may include SSRIs (e.g., citalopram, fluoxetine, sertraline, etc.), serotonin-norepinephrine reuptake inhibitors (e.g., duloxetine, venlafaxine, etc.), N-methyl-D-aspartate (NMDA) receptor modulators (e.g., ketamine, D-cycloserine, memantine, etc.), histone deacetylase inhibitors (e.g., valproic acid, b-hydroxybutyrate, etc.), serotonin 2A receptor agonists (e.g., N,N-dimethyltryptamine, lysergic acid diethylamide, psilocybin, etc.), entactogens (e.g., 3,4-methylenedioxymethamphetamine, 3,4-methylenedioxyamphetamine, etc.), lithium chloride, neurotrophic factors (e.g., BDNF, insulin-like growth factor 1, nerve growth factor, glial-derived growth factor, etc.), acetylcholine receptor modulators (e.g., donepezil, scopolamine, etc.) and others.

Selective serotonin reuptake inhibitors (SSRIs) are a class of drugs that are typically used as antidepressants in the treatment of major depressive disorder, anxiety disorders, and other psychological conditions. SSRIs increase the extracellular level of the neurotransmitter serotonin by limiting its reabsorption (reuptake) into the presynaptic cell. They have varying degrees of selectivity for the other monoamine transporters, with pure SSRIs having strong affinity for the serotonin transporter and only weak affinity for the norepinephrine and dopamine transporters. SSRIs include fluoxetine, paroxetine, sertraline, escitalopram and citalopram.

In an embodiment, administration of the compound induces neurite outgrowth. In an embodiment neurite outgrowth includes neurite number, neurite total length, number of neurite branch points per neuron or any combination thereof. In an embodiment, the neurite outgrowth includes neurite outgrowth on prefrontal cortex neurons and/or hippocampal neurons.

In some embodiments, the method comprises administering a therapeutic agent.

In some embodiments, the compound of Formula I, a derivative thereof, or a combination thereof, is administered prior to, simultaneously with or following administration of the therapeutic agent.

### Neurite Outgrowth

Signs of neuronal atrophy have been found in brain regions involved in stress-related behaviors, including prefrontal cortex and hippocampus. In animals, these structural changes have been shown to include loss of neurites, dendritic spines and synaptic contacts, as well as reduced hippocampal neurogenesis. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Thus, compounds that promote the generation and maintenance of neurites, spines, synapses and/or neurons upon single administration may have rapid therapeutic benefit in the treatment of stress-related disorders. Neurite outgrowth is measured by increasing neurite number, neurite total length and total number of branch points.

Some embodiments are directed towards a method of inducing neurite outgrowth in a subject in need thereof, comprising administering to the subject a therapeutically effective amount of a compound of Formula I, a derivative thereof, or a combination thereof.

In an embodiment, neurite outgrowth includes neurite number, neurite total length, number of neurite branch points per neuron or any combination thereof. In an embodiment, neurite outgrowth includes neurite outgrowth on prefrontal cortex neurons and/or hippocampal neurons. In an embodiment, the subject has a stress-related disease or disorder.

In some embodiments the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

In some embodiments, the method comprises administering a therapeutic agent.

In some embodiments, the compound of Formula I, a derivative thereof, or a combination thereof, is administered prior to, simultaneously with or following administration of the therapeutic agent.

### Neural Atrophy

It has been shown that chronic stress from conditions such as PTSD can cause neural atrophy and decrease the number of synapses within cortical and limbic circuits, which are associated with the regulation of mood, cognition, and behavior. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Neural plasticity, and therefore neural atrophy, can be improved by the induction of neurite growth. Therefore, compounds that promote neural plasticity and neurite growth may have therapeutic benefit in the treatment of stress-related disorders.

Some embodiments are directed towards a method of treating neuronal atrophy in a subject in need thereof, comprising administering to the subject a therapeutically effective amount of a compound of Formula I, a derivative thereof, or a combination thereof.

In an embodiment, administration of the compound induces neurite outgrowth. In an embodiment neurite outgrowth includes neurite number, neurite total length, number of neurite branch points per neuron or any combination thereof. In an embodiment, the neurite outgrowth includes neurite outgrowth on prefrontal cortex neurons and/or hippocampal neurons.

In an embodiment, the subject has a stress-related disease or disorder.

In some embodiments the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

In some embodiments, the method comprises administering a therapeutic agent.

In some embodiments, the compound of Formula I, a derivative thereof, or a combination thereof, is administered prior to, simultaneously with or following administration of the therapeutic agent.

### Neural Plasticity

Neuroplasticity is the ability of the brain to form and reorganize synaptic connections, especially in response to learning or experience or following an injury. It has been shown that exposure to stress causes a consistent suppression of neural plasticity. Therefore, traumatic events, such as events causing PTSD, can alter the neural connections and neural plasticity of the brain. However, the neuroplasticity can be used to mitigate the effects of PTSD. It has been shown that chronic, but not acute, administration of typical antidepressant drugs, such as selective serotonin reuptake inhibitors (SSRIs), attenuate the effects of stress on neurogenesis and neuronal structure in animals and demonstrate antidepressant and anxiolytic effects in humans. Neural plasticity can be improved by the induction of neurite growth. As such, compounds that promote neural plasticity may have therapeutic benefit in the treatment of stress-related disorders.

Some embodiments are directed towards a method of inducing structural neuroplasticity in a subject in need thereof, comprising administering to the subject a therapeutically effective amount of a compound of Formula I, a derivative thereof, or a combination thereof.

In an embodiment, the compound has antidepressant and anxiolytic effects.

In an embodiment, administration of the compound induces neurite outgrowth. In an embodiment neurite outgrowth includes neurite number, neurite total length, number of neurite branch points per neuron or any combination thereof. In an embodiment, the neurite outgrowth includes neurite outgrowth on prefrontal cortex neurons and/or hippocampal neurons.

In an embodiment, the subject has a stress-related disease or disorder.

In some embodiments the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

In some embodiments, the method comprises administering a therapeutic agent.

In some embodiments, the compound of Formula I, a derivative thereof, or a combination thereof, is administered prior to, simultaneously with or following administration of the therapeutic agent.

### EXAMPLES

### General Synthetic Experimental Details

Unless otherwise noted, all materials/reagents were obtained from commercial suppliers and used without further purification. Reactions were monitored by LCMS and/or thin layer chromatography (TLC) on silica gel 60 F254 (0.2 mm) pre-coated aluminum foil or glass-backed and visualized using UV light. ¹HNMR (400 MHZ) spectra was recorded on Broker spectrometers at RT with TMS or the residual solvent peak as the internal standard. The line positions or multiples are given in (8), and the coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened). Preparative HPLC purifications were performed on Shimadzu LC-6AD. All purification work was completed using a Shim-pack PREP-DDS (H) KIT Column. Preparative TLC was performed on Whatman LK6F Silica Gel 60A size 20×20 cm plates with a thickness of 1000 μm or equivalent. LCMS was performed on Shimadzu LCMS-2020 equipped with LC-20AD or 30AD pumps, SPD-M20APDA and Alltech 3300 ELSD. Mobile Phase A: water (0.1% formic acid); Mobile Phase B: acetonitrile (ACN); Duration: 5 minutes; Column: Sepax BR-C18 4.6*50 mm, 3 μm; Flow Rate: 1.0 mL/min; Oven Temperature: 40° C. Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. ¹HNMR (400 MHZ) spectra was recorded on Bruker spectrometers at room temperature (RT) with TMS or residual solvent peak as internal standard. The line positions or multiples are given in (δ) and coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened). Preparative HPLC purification was performed on Shimadzu LC-6AD. All purification work was complete using Shim-pack PREP-DDS (H) KIT column. The mobile phases were water (with 0.1% HCO2H) and acetonitrile. all reagents used were HPLC grade. The flow rate was 10 mL/min.

### EXAMPLES 1, 2 and 3

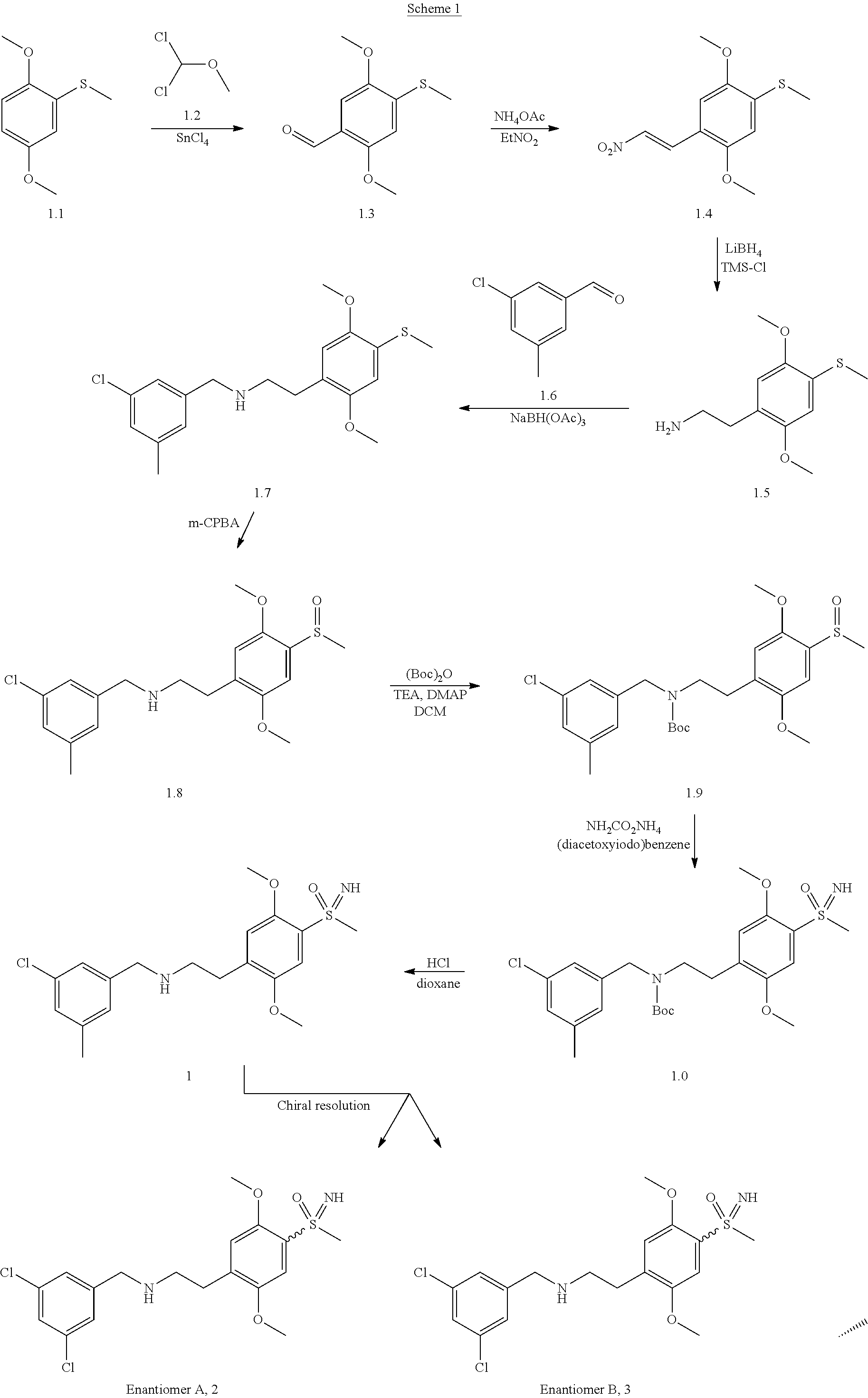

### Synthesis of: 2,5-dimethoxy-4-(methylthio)-benzaldehyde (1.3)

To a solution of 1.1 (1.26 g, 6.85 mmol, 1 eq) in DCM (15 mL) was added dichloro(methoxy) Methane, 1.2 (0.86 g, 7.54 mmol, 1.1 eq), followed by addition of SnCl₄ (3.57 g, 13.7 mmol, 2 eq) at 0° C. The resulting mixture was stirred at RT overnight. The reaction was quenched with water, and then extracted with ethyl acetate (3×50 mL). The combined organic layers were washed with brine, dried over Na₂SO₄, filtered, and concentrated to give a crude product which was purified by silica gel column chromatography (eluted with petroleum ether/ethyl acetate=1:1) to afford the title compound 1.3 as a colorless oil. (1.45 g, 100%) LCMS: m/z 213.30 [M+H]⁺. ¹H NMR (400 MHZ, DMSO-d₆) δ 10.24 (s, 1H), 7.12 (s, 1H), 6.88 (s, 1H), 3.94 (s, 3H), 3.81 (s, 3H), 2.52 (s, 3H).

### Synthesis (E)-(2,5-dimethoxy-4-(2-nitrovinyl)phenyl)(methyl) sulfane (1.4)

A suspension of 1.3 (300 mg, 1.42 mmol) and ammonium acetate (55 mg, 0.71 mmol) in nitromethane (10 mL) was stirred at 80° C. for 12 h and a yellow solid appeared. Water and EtOH (5 mL) were added, and the mixture was stirred for 30 min at RT. yellow solid was then filtered, washed by water (3×10 mL) and dried at 50° C. to afford the title compound 1.4 (385 mg, 22%) as a yellow solid. LCMS: m/z 255.90 [M+H]⁺ ¹H NMR (400 MHZ, Chloroform-d) δ 8.13 (d, J=13.5 Hz, 1H), 7.83 (d, J=13.6 Hz, 1H), 6.83 (s, 1H), 6.71 (s, 1H), 3.95 (s, 3H), 3.89 (s, 3H), 2.49 (s, 3H).

### Synthesis 2-(2,5-dimethoxy-4-(methylthio)phenyl) ethan-1-amine hydrochloride (1.5)

A suspension of LiBH₄ (109 mg, 5.01 mmol) in 10 mL THF was cooled to 0° C. under nitrogen. TMSCl (1.08 g, 10.0 mmol) was added slowly to the solution at 0° C. After the addition was complete, the mixture was stirred 30 min at 0° C. Intermediate 1.4 (319 mg, 1.25 mmol) dissolved in THF (5 mL) and was added to the reaction. The resulting mixture was heated to reflux for 4 h and water (20 mL) was added to quench the LiBH₄, followed by addition of Na₂CO₃ to adjust pH to 11. The resulting mixture was extracted with DCM (3×15 mL). The combined organic layers were collected, dried over Na₂SO₄ and concentrated under reduced pressure to afford the crude product which was purified by silica gel flash chromatography (Eluted with DCM/CH₃OH=20:1) to afford the title compound 1.5 (272 mg, 83%) as a white solid after treatment with HCl in dioxane. LCMS: m/z 228.40 [M+H]⁺. ¹H NMR (400 MHZ, DMSO-d6) δ 6.77 (s, 1H), 6.73 (s, 1H), 3.76 (s, 3H), 3.74 (s, 3H), 2.68 (d, J=7.7 Hz, 2H) 2.59 (d, J=7.7 Hz, 2H), 2.39 (s, 3H).

### Synthesis of N-(3-chloro-5-methylbenzyl)-2-(2,5-dimethoxy-4-(methylthio)phenyl)-ethan-1-amine hydrochloride (1.7)

To a stirred solution of 1.5 (202 mg, 0.77 mmol) and 3-chloro-5-methylbenzaldehyde, 1.6 (118 mg, 0.77 mmol) in dichloromethane (5 mL) was added NaBH(OAc)₃ (488 mg, 2.3 mmol). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by silica gel flash chromatography (eluted with petroleum ether/ethyl acetate=1:1) to afford the title compound 1.7, which was converted to the HCl salt (111 mg, 36%) as a white solid after treatment with HCl in dioxane. LCMS: m/z 366.35 [M+H]0.1H NMR (400 MHZ, Methanol-d₄) δ 7.40-7.31 (m, 2H), 7.25 (s, 1H), 6.84 (d, J=5.6 Hz, 2H), 4.20 (s, 2H), 3.86 (s, 3H), 3.84 (s, 3H), 3.29-3.21 (m, 2H), 3.05-2.97 (m, 2H), 2.44 (s, 3H), 2.41 (s, 3H).

### Synthesis of N-(3-chloro-5-methylbenzyl)-2-(2,5-dimethoxy-4-(methylsulfinyl)phenyl) ethan-1-amine (1.8)

To a stirred solution of 1.7 (50 mg, 0.14 mmol) in DCM (5 mL) was added m-CPBA (28 mg, 0.14 mmol) at 0° C. The resulting mixture was stirred at RT overnight. The reaction mixture was quenched with aqueous Na₂S₂O₄ and extracted with ethyl acetate. The organic layer was dried over anhydrous sodium sulfate and concentrated under reduced pressure to give a crude residue which was purified by silica gel flash chromatography (eluted with petroleum ether/ethyl acetate=1:1) to afford the title compound 1.8 which was converted to the HCl salt (24 mg, 42%) as a white solid after treatment with HCl in dioxane. LCMS: m/z 382.25 [M+H]⁺ ¹HNMR (400 MHz, Methanol-d₄) δ 7.38-7.33 (m, 3H), 7.29-7.23 (m, 1H), 7.05 (s, 1H), 4.22 (s, 2H), 3.92 (s, 3H), 3.90 (s, 3H), 3.32-3.26 (m, 2H), 3.14-3.07 (m, 2H), 2.81 (s, 3H), 2.41 (s, 3H).

### Synthesis of tert-butyl (3-chloro-5-methylbenzyl) (2,5-dimethoxy-4-(methylsulfinyl) phenethyl)-carbamate (1.9)

To a solution of N-(3-chloro-5-methylbenzyl)-2-(2,5-dimethoxy-4-(methylsulfinyl)-phenyl) ethan-1-amine 1.8 (283 mg, 0.74 mmol) in DCM was added di-tert-butyl dicarbonate (324 mg, 1.48 mmol), TEA (224 mg, 2.22 mmol) and DMAP (9 mg, 0.074 mmol). The resulting mixture was stirred at RT for 16 h. The reaction was quenched by water and extracted by DCM. The organic phase was concentrated under reduced pressure to afford the title compound 1.9 (330 mg, 92%), which was used directly for the next step without further characterization.

### Synthesis of tert-Butyl (3-chloro-5-methylbenzyl) (2,5-dimethoxy-4-(S-methylsulfon-imidoyl)-phenethyl) Carbamate (1.0)

tert-butyl (3-chloro-5-methylbenzyl) (2,5-dimethoxy-4-(methylsulfinyl) phenethyl)-carbamate 1.9 (180 mg, 0.37 mmol) in MeOH (5 mL) was added ammonium carbamate (58 mg, 0.74 mmol) and (diacetoxyiodo)benzene (238 mg, 0.74 mmol). The resulting mixture was stirred at RT for 16 h. The reaction was quenched with water and extracted by DCM. The organic phase was concentrated under reduced pressure to afford the crude product which was purified by flash chromatography to afford the title compound, 1.0 (100 mg, 52%).

### Synthesis of (4-(2-((3-Chloro-5-methylbenzyl)amino)ethyl)-2,5-dimethoxyphenyl)-(imino)-(methyl)-λ⁶-sulfanone hydrochloride (1)

tert-butyl (3-chloro-5-methylbenzyl) (2,5-dimethoxy-4-(S-methylsulfonimidoyl)-phenethyl)-carbamate 1.0 (100 mg, 0.20 mmol, 1 eq) in DCM (10 mL) was added 4 M HCl in dioxane (5 mL). The resulting mixture was stirred at RT for 16 h. The reaction mixture was concentrated, washed with petroleum ether (10 mL) and filtered to afford the title compound, 1 as the HCl salt (96 mg, 90%). LCMS: m/z 398.15 [M+H]⁺. ¹H NMR (400 MHZ, CDCl3) δ 7.46 (s, 1H), 7.09 (s, 1H), 7.05 (s, 1H), 6.98 (s, 1H), 6.89 (s, 1H), 3.94 (s, 3H), 3.83 (s, 3H), 3.76 (s, 2H), 3.28 (s, 3H), 2.87 (s, 4H), 2.31 (s, 3H).

### Compounds 2 (Enantiomer A) and Compound 3 (Enantiomer B)

Racemic (4-(2-((3-chloro-5-methylbenzyl)amino)ethyl)-2,5-dimethoxyphenyl) (imino)-(methyl)-16-sulfanone hydrochloride (60 mg, 0.14 mmol) was resolved by chiral chromatography to afford Compound 2 Enantiomer A (R) or(S)-(4-(2-((3-chloro-5-methylbenzyl)amino)ethyl)-2,5-dimethoxyphenyl) (imino)-(methyl)-λ⁶-sulfanone (18 mg) ¹H NMR (400 MHZ, CDCl₃) δ 7.46 (s, 1H), 7.09 (s, 1H), 7.05 (s, 1H), 6.98 (s, 1H), 6.89 (s, 1H), 3.93 (s, 3H), 3.83 (s, 3H), 3.75 (s, 2H), 3.28 (s, 3H), 2.87 (s, 4H), 2.31 (s, 3H). LCMS: m/z 398.15 [M+H]. Compound 3 Enantiomer B(R) or(S) (4-(2-((3-chloro-5-methylbenzyl)amino)ethyl)-2,5-dimethoxyphenyl) (imino)(methyl)-λ⁶-sulfanone. ¹H NMR (400 MHZ, CDCl3) δ 7.46 (s, 1H), 7.09 (s, 1H), 7.05 (s, 1H), 6.98 (s, 1H), 6.89 (s, 1H), 3.93 (s, 3H), 3.83 (s, 3H), 3.75 (s, 2H), 3.28 (s, 3H), 2.87 (s, 4H), 2.31 (s, 3H). LCMS: m/z 398.15 [M+H].

### EXAMPLES 4, 5 and 6

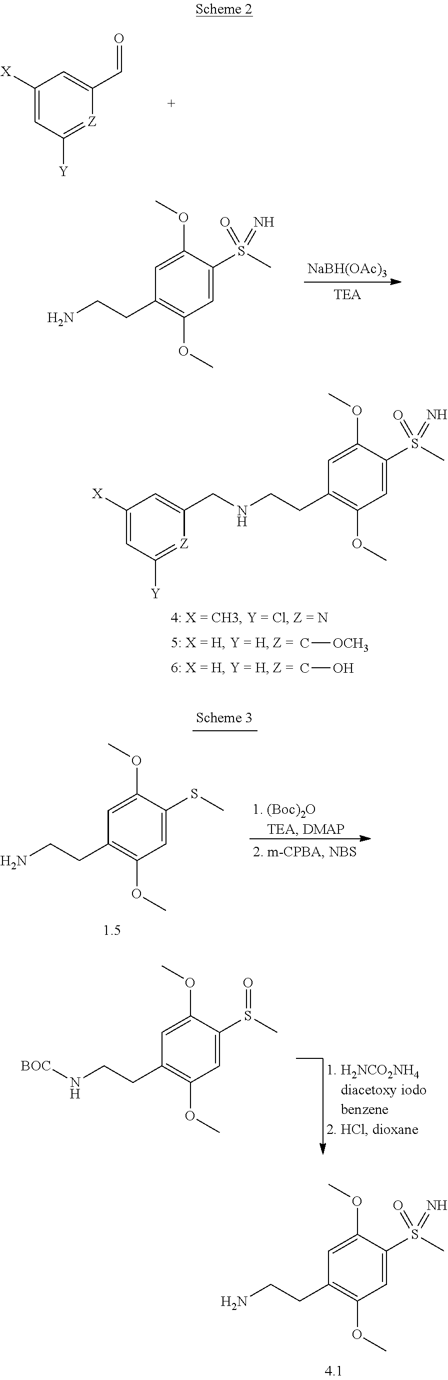

### (4-(2-(((6-chloro-4-methylpyridin-2-yl)methyl)amino)ethyl)-2,5-dimethoxypheny)(imino-)-(methyl)-λ⁶-sulfanone hydrochloride (4)

To a stirred solution of (4-(2-aminoethyl)-2,5-dimethoxyphenyl)-(imino)(methyl)-λ⁶-sulfanone hydrochloride (4.1) (50.0 mg, 0.17 mmol), which was prepared analogously to 1.0 as shown in Scheme 3, and 6-chloro-4-methylpicolin-aldehyde (26 mg, 0.17 mmol, 1 eq) in dichloromethane (5 mL) was added Et₃N (0.05 mL, 0.34 mmol, 2 eq) and NaBH(OAc)₃ (107 mg, 0.51 mmol, 3.0 eq). The resulting mixture was stirred at RT overnight. The reaction mixture was quenched with aqueous sodium bicarbonate solution, extracted with ethyl acetate and the organic layer was dried over anhydrous sodium sulfate. The dried solution was concentrated under reduced pressure to give a crude residue which was purified by flash chromatography (eluted with petroleum ether/ethyl acetate=1:1) to afford the title compound which was converted to the HCl salt (55 mg, 74%) as a white solid after treatment with HCl in dioxane. LCMS: (ES+): 397.85 m/z [M+H]⁺. ¹H NMR (400 MHZ, CD₃OD) δ 7.50 (s, 1H), 7.41 (s, 1H), 7.35 (s, 1H), 7.30 (s, 1H), 4.38 (s, 2H), 4.07 (s, 3H), 3.95 (s, 3H), 3.76 (s, 3H), 3.39 (t, J=7.6 Hz, 2H), 3.25-3.20 (m, 2H), 2.41 (s, 3H).

### (2,5-dimethoxy-4-(2-((2-methoxybenzyl)amino)ethyl)phenyl) (imino)(methyl)-λ⁶-sulfanone hydrochloride (5)

To a stirred solution of (4-(2-aminoethyl)-2,5-dimethoxyphenyl) (imino)(methyl)-λ⁶-sulfanone hydrochloride (50.0 mg, 0.17 mmol) and 2-methoxybenzaldehyde (22 mg, 0.17 mmol, 1 eq) in dichloromethane (5 mL) was added Et₃N (0.05 mL, 0.34 mmol, 2 eq) and NaBH(OAc) 3 (107 mg, 0.51 mmol, 3.0 eq). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by flash chromatography (eluted with petroleum ether/EA=1:1) to afford the title compound which was converted to the HCl salt (30 mg, 42%) as a white solid after treatment with HCl in dioxane. LCMS: (ES+): 379.35 m/z [M+H]. ¹H NMR (400 MHZ, CDCl3) δ 7.38-7.34 (m, 2H), 7.28 (s, 1H), 7.11 (s, 1H), 6.94 (t, J=7.3 Hz, 1H), 6.89 (d, J=8.4 Hz, 1H), 4.19-4.08 (m, 2H), 3.94 (s, 3H), 3.81 (s, 3H), 3.79 (s, 3H), 3.58 (s, 3H), 3.25-3.15 (m, 2H), 3.15-3.02 (m, 2H).

### (4-(2-((2-hydroxybenzyl)amino)ethyl)-2,5-dimethoxyphenyl) (imino)(methyl)-λ⁶-sulfanone hydrochloride (6)

To a stirred solution of (4-(2-aminoethyl)-2,5-dimethoxyphenyl) (imino)(methyl)-λ⁶-sulfanone hydrochloride (50 mg, 0.17 mmol), and 2-hydroxybenzaldehyde (20 mg, 0.17 mmol, 1 eq) in dichloromethane (5 mL) was added Et₃N (0.05 mL, 0.34 mmol, 2 eq) and NaBH(OAc) 3 (107 mg, 0.51 mmol, 3.0 eq). The resulting mixture was stirred at RT overnight. The reaction mixture was quenched with aqueous sodium bicarbonate solution and extracted with ethyl acetate. The organic layer was dried over anhydrous sodium sulfate and concentrated under reduced pressure to give a crude residue which was purified by flash chromatography (eluted with petroleum/EA=1:1) to afford the title compound which was converted to the HCl salt (37 mg, 54%) as a white solid after treatment with HCl in dioxane. LCMS: (ES+): 365.10 m/z [M+H]⁺. ¹H NMR (400 MHZ, CD₃OD) δ 7.48 (s, 1H), 7.42 (s, 1H), 7.36-7.27 (m, 2H), 6.94-6.89 (m, 2H), 4.27 (s, 2H), 4.07 (s, 3H), 3.91 (s, 3H), 3.85 (s, 3H), 3.30-3.26 (m, 2H), 3.22-3.17 (m, 2H).

### Example 2

### GPCR Arrestin Assay Methods

### Arrestin Pathway (Performed by DiscoverX Eurofins)

The PathHunter® β-Arrestin assay monitors the activation of a GPCR in a homogenous, non-imaging assay format using a technology developed by DiscoverX called Enzyme Fragment Complementation (EFC) with β-galactosidase (β-Gal) as the functional reporter. The enzyme is split into two inactive complementary portions (EA for Enzyme Acceptor and PK for ProLink) expressed as fusion proteins in the cell. EA is fused to β-Arrestin and PK is fused to the GPCR of interest. When the GPCR is activated and β-Arrestin is recruited to the receptor, ED and EA complementation occurs, restoring β-Gal activity which is measured using chemiluminescent PathHunter® Detection Reagents. PathHunter cell lines (DiscoveRx Eurofins) were expanded from freezer stocks according to standard procedures. Cells were seeded in a total volume of 20 μL into white walled, 384-well microplates and incubated at 37° C. for the appropriate time prior to testing. For agonist determination, cells were incubated with sample to induce response. Intermediate dilution of sample stocks was performed to generate 5× sample in assay buffer. 5 μL of 5× sample was added to cells and incubated at 37° C. or room temperature for 90 to 180 minutes. Vehicle concentration was 1%. β-Arrestin assay signal was generated through a single addition of 12.5 or 15 μL (50% v/v) of PathHunter Detection reagent cocktail, followed by a one-hour incubation at room temperature. Microplates were read following signal generation with a PerkinElmer Envision™ instrument for chemiluminescent signal detection. Compound activity was analyzed using CBIS data analysis suite (ChemInnovation, CA). For agonist mode assays, percentage activity was calculated using the following formula:

% Activity=100%×(mean RLU of test sample−mean RLU of vehicle control)/(mean MAX control ligand−mean RLU of vehicle control).

In these studies, the MAX control ligand response was generated using 1-10 μM serotonin.

### Calcium Mobilization Assay Method (Performed by DiscoveRx Eurofins)

The Calcium No-Wash^{PLUS} assay monitors the activation of a GPCR via Gq secondary messenger signaling in a live cell, non-imaging assay format. Calcium mobilization in PathHunter® cell lines or other cell lines stably expressing Gq-coupled GPCRs is monitored using a calcium-sensitive dye that is loaded into cells. GPCR activation by a compound result in the release of calcium from intracellular stores and an increase in dye fluorescence that is measured in real-time. Cell lines expressing the GPCR of interest were expanded from freezer stocks according to standard procedures. Cells were seeded in a total volume of 20 μL into black-walled, clear-bottom, Poly-D-lysine coated 384-well microplates and incubated at 37° C. for the appropriate time prior to testing. Assays were performed in 1×Dye Loading Buffer consisting of 1× Dye, 1× Additive A and 2.5 mM Probenecid in HBSS/20 mM Hepes. Probenicid was prepared fresh. Cells were loaded with dye prior to testing. Media was aspirated from cells and replaced with 20 μL Dye Loading Buffer. Cells were incubated for 30-60 minutes at 37° C. For agonist determination, cells were incubated with sample to induce response. After dye loading, cells were removed from the incubator and 10 μL HBSS/20 mM Hepes was added. 3× vehicle was included in the buffer when performing agonist dose curves to define the EC80 for subsequent antagonist assays. Cells were incubated for 30 minutes at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay. Compound activity data was analyzed using CBIS data analysis suite (ChemInnovation, CA). For agonist mode assays, percentage activity is calculated using the following formula:

% Activity=100%×(mean RFU of test sample−mean RFU of vehicle control)/(mean MAX RFU control ligand−mean RFU of vehicle control).

In these studies, the MAX RFU was generated by using 0.1 μM serotonin for the calcium mobilization assay.

Results are shown in Table 1

TABLE 1

| | 5-HT2A receptor, | 5-HT2A | 5-HT2C | 5-HT2B |
|---|---|---|---|---|
| | Ca⁺⁺ Flux | receptor, | receptor, | receptor, |
| | EC₅₀ (μM)/ | β-arrestin2 | Ca⁺⁺ Flux | Ca⁺⁺ Flux |
| No | % activation @30 μM | EC₅₀ (μM) | EC₅₀ (μM) | EC₅₀ (μM) |
| 1 | 0.0041/102 | 0.0250 | NA | >1 |
| 2 | NA/57 | NA | NA | NA |
| 3 | 0.0024/78 | 0.0136 | 1.490 | >1 |
| 4 | NA/67 | NA | NA | NA |
| 5 | 0.0017/111 | 0.0032 | 0.0036 | NA |
| 6 | NA/ 82 | NA | NA | NA |

Although the invention has been described with reference to the above examples, it will be understood that modifications and variations are encompassed within the spirit and scope of the invention. Accordingly, the invention is limited only by the following claims.

1. A compound, or a derivative thereof, of Formula I: 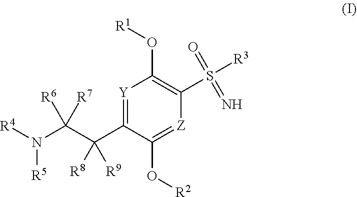
wherein:
R¹ and R² are independently H, D, C₁-C₆ alkyl, or C₁-C₆ fluoroalkyl;
R³ is H, C₁-C₆ alkyl, C₁-C₆ fluoroalkyl, CH₂X, CHX₂, or CX₃ wherein the alkyl or fluoroalkyl is optionally substituted with one or more D;
R⁴ is H, C₁-C₆ alkyl, or C₁-C₆ fluoroalkyl wherein the alkyl or fluoroalkyl is optionally substituted with a phenyl or a C₁-C₅ heteroaryl and wherein the phenyl or heteroaryl is optionally substituted with one or more of D, a halogen, or a C₁-C₆ alkyl;
R⁵ is H, a C₁-C₆ alkyl, or a C₁-C₆ fluoroalkyl;
R⁶, R⁷, R⁸, and R⁹ are independently H, D, a C₁-C₆ alkyl, a C₁-C₆ fluoroalkyl, a halogen, a C₁-C₇ heterocycloalkyl, a C₃-C₈ cycloalkyl, a C₁-C₇ heteroaryl, —OH, or —OD; or
R⁶ and R⁷ or R⁸ and R⁹ are independently taken together to form ═O;
X is at each instance is independently F, Cl, Br, or I;
Y is CR¹⁰ or N;
Z is CR¹¹ or N;
R¹⁰ and R¹¹ are independently H, D, CN, a halogen, OH, OD, OCH₃, CF₃, or COOR¹²; and
R¹² is H, a C₁-C₆ alkyl, a C₁-C₆ fluoroalkyl, or a C₁-C₇ heterocycloalkyl.

2. The compound of claim 1, wherein the compound, or derivative thereof, is:

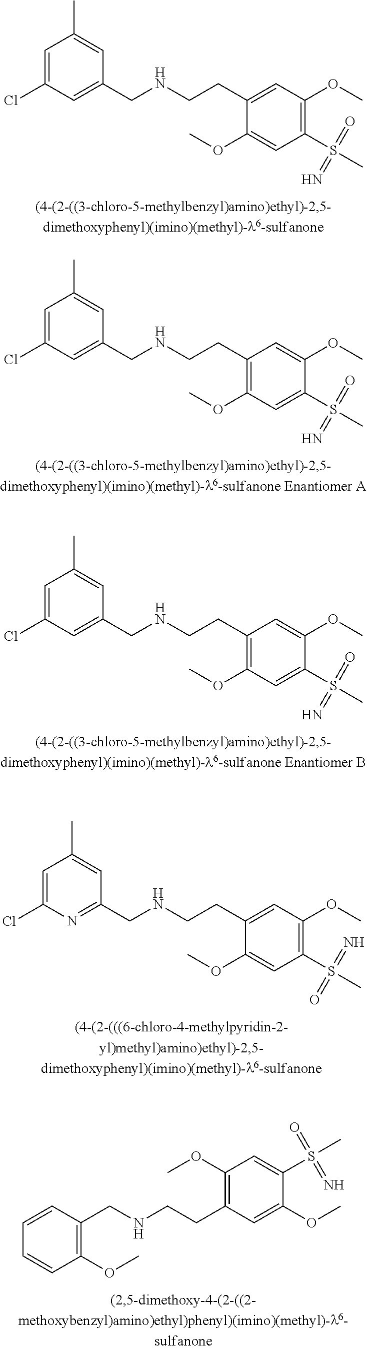 [SMILES: COc1cc(S(C)(=N)=O)c(OC)cc1CCNCc1cc(C)cc(Cl)c1 ; COc1cc(S(C)(=N)=O)c(OC)cc1CCNCc1cc(C)cc(Cl)c1 ; COc1cc(S(C)(=N)=O)c(OC)cc1CCNCc1cc(C)cc(Cl)c1 ; COc1cc(S(C)(=N)=O)c(OC)cc1CCNCc1cc(C)cc(Cl)n1 ; COc1cc(S(C)(=N)=O)c(OC)cc1CCNCc1ccccc1OC]

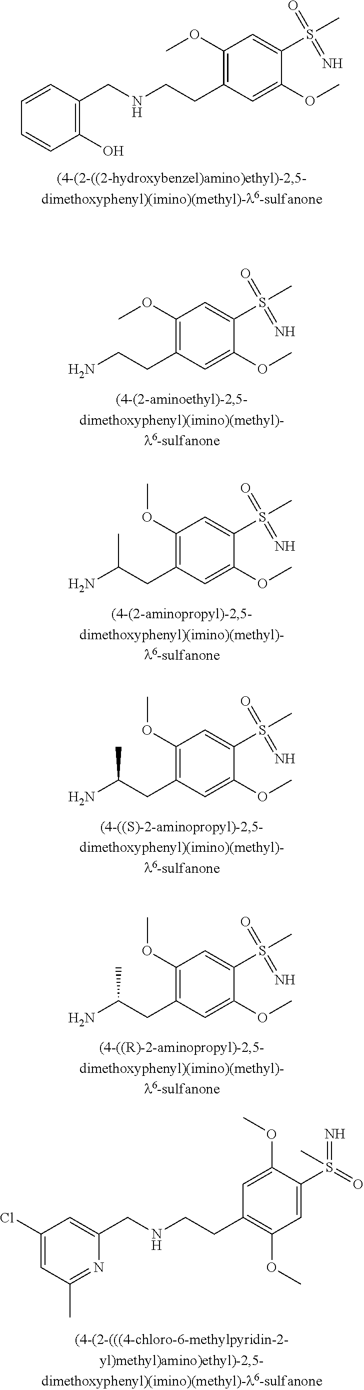 [SMILES: COc1cc(S(C)(=N)=O)c(OC)cc1CCNCc1ccccc1O ; COc1cc(S(C)(=N)=O)c(OC)cc1CCN ; COc1cc(S(C)(=N)=O)c(OC)cc1CC(C)N ; COc1cc(S(C)(=N)=O)c(OC)cc1C[C@H](C)N ; COc1cc(S(C)(=N)=O)c(OC)cc1C[C@@H](C)N ; COc1cc(S(C)(=N)=O)c(OC)cc1CCNCc1cc(Cl)cc(C)n1]

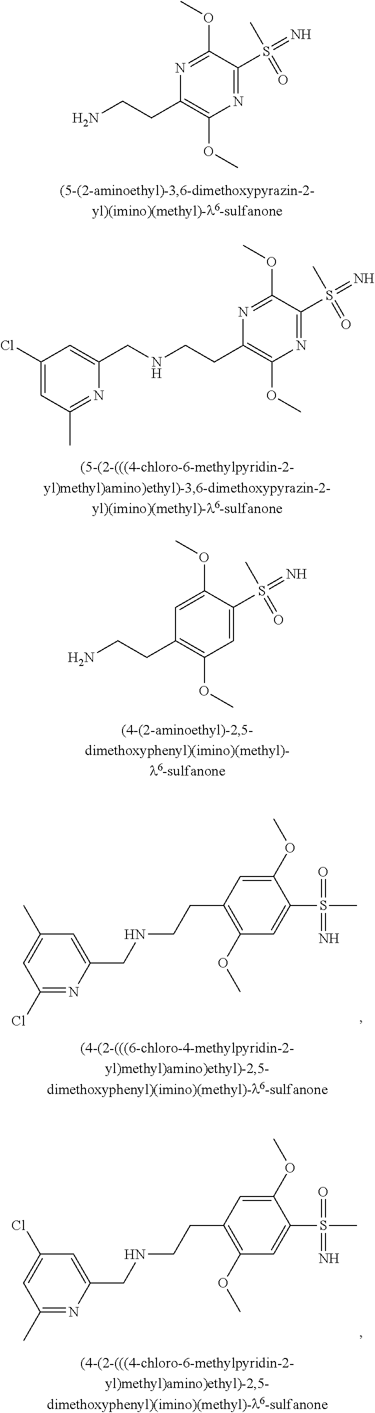 [SMILES: COc1nc(S(C)(=N)=O)c(OC)nc1CCN ; COc1nc(S(C)(=N)=O)c(OC)nc1CCNCc1cc(Cl)cc(C)n1 ; COc1cc(S(C)(=N)=O)c(OC)cc1CCN ; COc1cc(S(C)(=N)=O)c(OC)cc1CCNCc1cc(C)cc(Cl)n1 ; COc1cc(S(C)(=N)=O)c(OC)cc1CCNCc1cc(Cl)cc(C)n1]

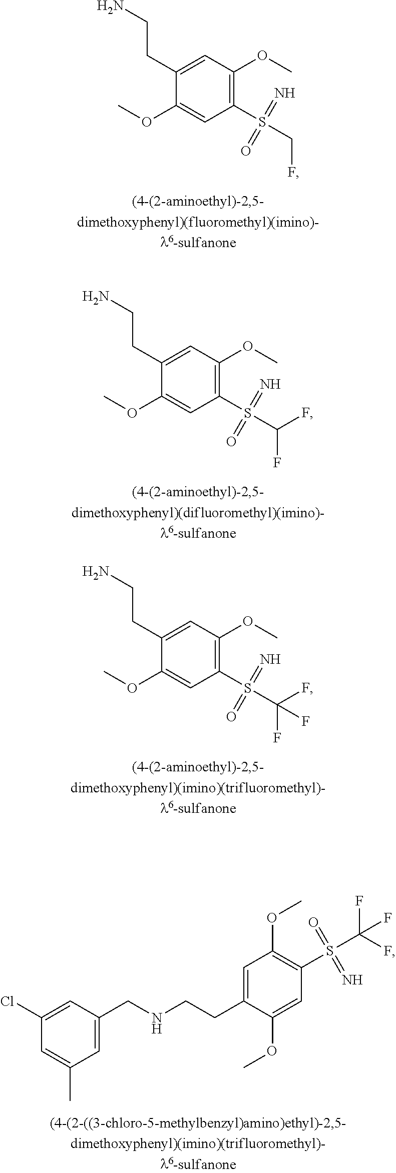 [SMILES: COc1cc(S(=N)(=O)CF)c(OC)cc1CCN ; COc1cc(S(=N)(=O)C(F)F)c(OC)cc1CCN ; COc1cc(S(=N)(=O)C(F)(F)F)c(OC)cc1CCN ; COc1cc(S(=N)(=O)C(F)(F)F)c(OC)cc1CCNCc1cc(C)cc(Cl)c1]

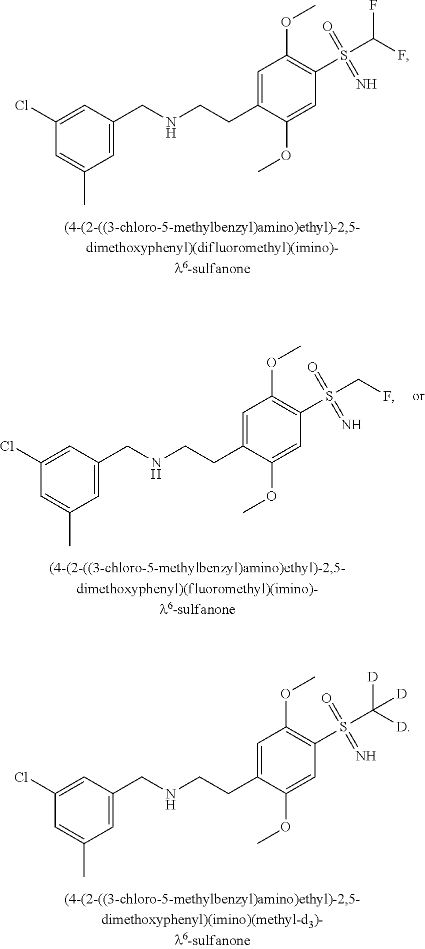

3. The compound of claim 1, wherein the compound is (4-(2-((3-chloro-5-methylbenzyl)amino)ethyl)-2,5-dimethoxyphenyl) (imino)(methyl)-λ⁶-sulfanone:

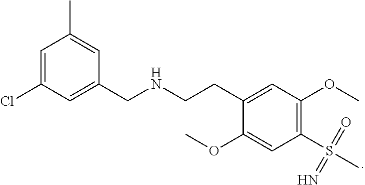 [SMILES: COc1cc(S(C)(=N)=O)c(OC)cc1CCNCc1cc(C)cc(Cl)c1]

4. The compound of claim 1, wherein the compound is (4-(2-((3-chloro-5-methylbenzyl)amino)ethyl)-2,5-dimethoxyphenyl) (imino)(methyl)-λ⁶-sulfanone Enantiomer A:

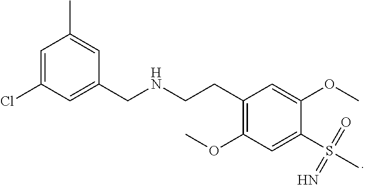 [SMILES: COc1cc(S(C)(=N)=O)c(OC)cc1CCNCc1cc(C)cc(Cl)c1]

5. The compound of claim 1, wherein the compound is (4-(2-((3-chloro-5-methylbenzyl)amino)ethyl)-2,5-dimethoxyphenyl) (imino)(methyl)-λ⁶-sulfanone Enantiomer B:

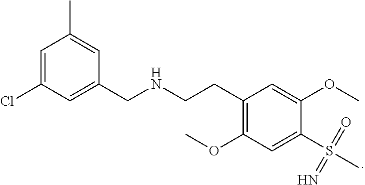 [SMILES: COc1cc(S(C)(=N)=O)c(OC)cc1CCNCc1cc(C)cc(Cl)c1]

6. The compound of claim 1, wherein the compound is (4-(2-(((6-chloro-4-methylpyridin-2-yl)methyl)amino)ethyl)-2,5-dimethoxyphenyl) (imino)(methyl)-λ⁶-sulfanone:

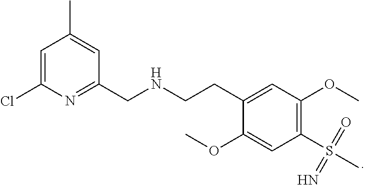 [SMILES: COc1cc(S(C)(=N)=O)c(OC)cc1CCNCc1cc(C)cc(Cl)n1]

7. The compound of claim 1, wherein the compound is (2,5-dimethoxy-4-(2-((2-methoxybenzyl)amino)ethyl)phenyl) (imino)(methyl)-λ⁶-sulfanone:

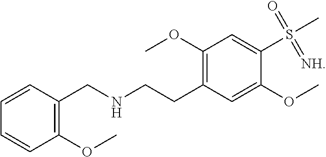 [SMILES: COc1cc(S(C)(=N)=O)c(OC)cc1CCNCc1ccccc1OC]

8. The compound of claim 1, wherein the compound is (4-(2-((2-hydroxybenzyl)amino)ethyl)-2,5-dimethoxyphenyl) (imino)(methyl)-λ⁶-sulfanone:

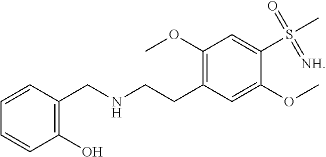 [SMILES: COc1cc(S(C)(=N)=O)c(OC)cc1CCNCc1ccccc1O]

9. The compound of claim 1, wherein the compound is (4-(2-aminoethyl)-2,5-dimethoxyphenyl) (imino)(methyl)-λ⁶-sulfanone:

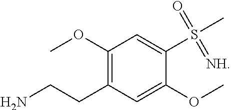 [SMILES: COc1cc(S(C)(=N)=O)c(OC)cc1CCN]

10. The compound of claim 1, wherein the compound is (4-(2-aminopropyl)-2,5-dimethoxyphenyl) (imino)(methyl)-λ⁶-sulfanone:

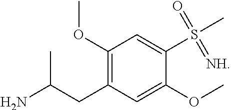 [SMILES: COc1cc(S(C)(=N)=O)c(OC)cc1CC(C)N]

11. The compound of claim 1, wherein the compound is (4-((S)-2-aminopropyl)-2,5-dimethoxyphenyl) (imino)(methyl)-λ⁶-sulfanone:

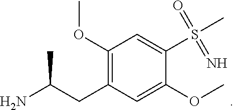 [SMILES: COc1cc(S(C)(=N)=O)c(OC)cc1C[C@H](C)N]

12. The compound of claim 1, wherein the compound is (4-((R)-2-aminopropyl)-2,5-dimethoxyphenyl) (imino)(methyl)-6-sulfanone:

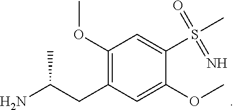 [SMILES: COc1cc(S(C)(=N)=O)c(OC)cc1C[C@@H](C)N]

13. The compound of claim 1, wherein the compound is (4-(2-(((4-chloro-6-methylpyridin-2-yl)methyl)amino)ethyl)-2,5-dimethoxyphenyl) (imino)(methyl)-λ⁶-sulfanone:

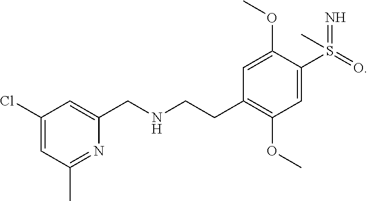 [SMILES: COc1cc(S(C)(=N)=O)c(OC)cc1CCNCc1cc(Cl)cc(C)n1]

14. The compound of claim 1, wherein the compound is (5-(2-aminoethyl)-3,6-dimethoxypyrazin-2-yl) (imino)(methyl)-λ⁶-sulfanone:

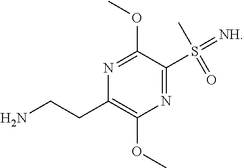 [SMILES: COc1nc(S(C)(=N)=O)c(OC)nc1CCN]

15. The compound of claim 1, wherein the compound is (5-(2-(((4-chloro-6-methylpyridin-2-yl)methyl)amino)ethyl)-3,6-dimethoxypyrazin-2-yl) (imino)(methyl)-λ⁶-sulfanone:

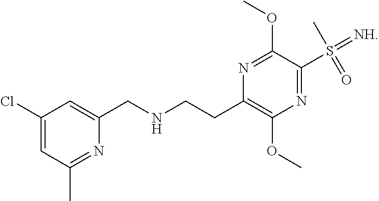 [SMILES: COc1nc(S(C)(=N)=O)c(OC)nc1CCNCc1cc(Cl)cc(C)n1]

16. The compound of claim 1, wherein the compound is (4-(2-aminoethyl)-2,5-dimethoxyphenyl) (imino)(methyl)-λ⁶-sulfanone:

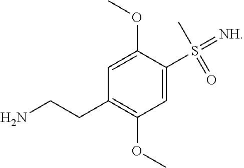 [SMILES: COc1cc(S(C)(=N)=O)c(OC)cc1CCN]

17. The compound of claim 1, wherein the compound is (4-(2-(((6-chloro-4-methylpyridin-2-yl)methyl)amino)ethyl)-2,5-dimethoxyphenyl) (imino)(methyl)-λ⁶-sulfanone:

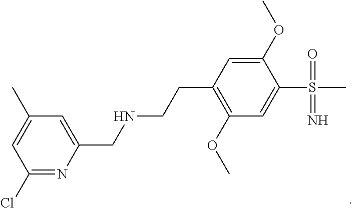 [SMILES: COc1cc(S(C)(=N)=O)c(OC)cc1CCNCc1cc(C)cc(Cl)n1]

18. The compound of claim 1, wherein the compound is (4-(2-(((4-chloro-6-methylpyridin-2-yl)methyl)amino)ethyl)-2,5-dimethoxyphenyl) (imino)(methyl)-λ⁶-sulfanone:

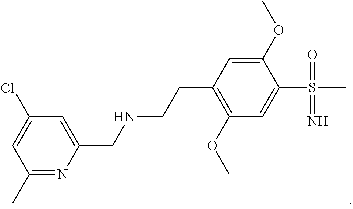 [SMILES: COc1cc(S(C)(=N)=O)c(OC)cc1CCNCc1cc(Cl)cc(C)n1]

19. The compound of claim 1, wherein the compound is (4-(2-aminoethyl)-2,5-dimethoxyphenyl) (fluoromethyl) (imino)-λ⁶-sulfanone:

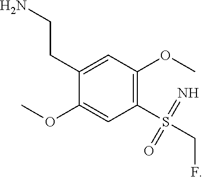 [SMILES: COc1cc(S(=N)(=O)CF)c(OC)cc1CCN]

20. The compound of claim 1, wherein the compound is (4-(2-aminoethyl)-2,5-dimethoxyphenyl) (difluoromethyl) (imino)-λ⁶-sulfanone:

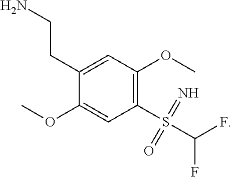 [SMILES: COc1cc(S(=N)(=O)C(F)F)c(OC)cc1CCN]

21. The compound of claim 1, wherein the compound is (4-(2-aminoethyl)-2,5-dimethoxyphenyl) (imino) (trifluoromethyl)-λ⁶-sulfanone:

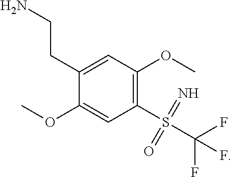 [SMILES: COc1cc(S(=N)(=O)C(F)(F)F)c(OC)cc1CCN]

22. The compound of claim 1, wherein the compound is (4-(2-((3-chloro-5-methylbenzyl)amino)ethyl)-2,5-dimethoxyphenyl) (imino) (trifluoromethyl)-λ⁶-sulfanone:

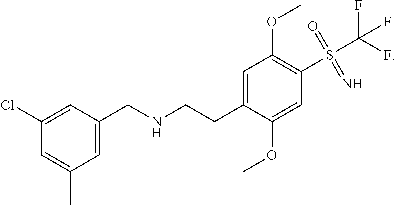 [SMILES: COc1cc(S(=N)(=O)C(F)(F)F)c(OC)cc1CCNCc1cc(C)cc(Cl)c1]

23. The compound of claim 1, wherein the compound is (4-(2-((3-chloro-5-methylbenzyl)amino)ethyl)-2,5-dimethoxyphenyl) (difluoromethyl) (imino)-λ⁶-sulfanone:

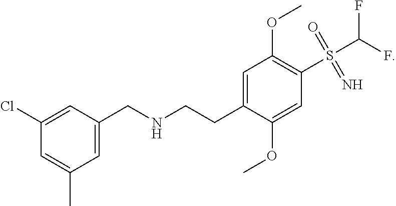 [SMILES: COc1cc(S(=N)(=O)C(F)F)c(OC)cc1CCNCc1cc(C)cc(Cl)c1]

24. The compound of claim 1, wherein the compound is (4-(2-((3-chloro-5-methylbenzyl)amino)ethyl)-2,5-dimethoxyphenyl) (fluoromethyl) (imino)-λ⁶-sulfanone:

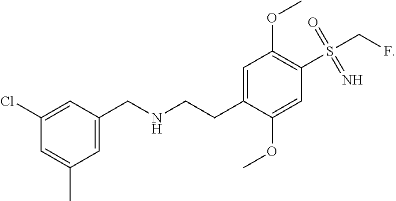 [SMILES: COc1cc(S(=N)(=O)CF)c(OC)cc1CCNCc1cc(C)cc(Cl)c1]

25. The compound of claim 1, wherein the compound is (4-(2-((3-chloro-5-methylbenzyl)amino)ethyl)-2,5-dimethoxyphenyl) (imino)(methyl-d₃)-λ⁶-sulfanone:

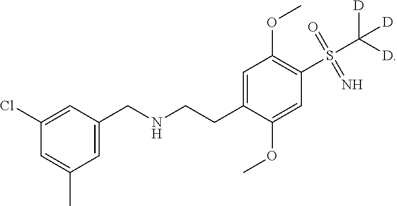

26. A composition comprising: a compound of claim 1, a derivative thereof, or a combination thereof; and a pharmaceutically acceptable excipient.

27. A method of treating a stress-related disease or disorder in a subject in need thereof, comprising administering to the subject a therapeutically effective amount of a compound of claim 1, a derivative thereof, or a combination thereof.

28. The method of claim 27, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

29. The method of claim 28, wherein the stress-related disease or disorder is depression, anxiety, or post-traumatic stress disorder.

30. A method for inducing neurite outgrowth in a subject in need thereof, comprising administering to the subject a therapeutically effective amount of a compound of claim 1, a derivative thereof, or a combination thereof.

31. A method of treating neuronal atrophy in a subject in need thereof, comprising administering to the subject a therapeutically effective amount of a compound of claim 1, a derivative thereof, or a combination thereof.

32. A method of inducing structural neuroplasticity in a subject in need thereof, comprising administering to the subject a therapeutically effective amount of a compound of claim 1, a derivative thereof, or a combination thereof.

33. The method of claim 30, wherein the subject has a stress-related disease or disorder.

34. The method of claim 33, wherein the stress-related disease or disorder is mood/depressive disorder, bipolar disorder, anxiety disorder, psychotic or delirium disorder, schizophrenia, schizoaffective disorder, personality disorder, abuse or neglect disorder, tic disorder, neurocognitive disorder, neurodevelopmental disorder, learning disorder, disruptive mood regulation disorder, intermittent explosive disorder, antisocial personality disorder, conduct disorder, behavioral and psychological symptoms of dementia, depression, anxiety, post-traumatic stress disorder (PTSD), major depressive disorder (MDD), treatment-resistant depression (TRD), substance use disorder (SUD), compulsive disorders, stress disorders, rumination, eating disorders, or a combination thereof.

35. The method of claim 27, wherein the compound has antidepressant and anxiolytic effects.

36. The method of claim 27, wherein the administering comprises intracutaneous, subcutaneous, intravenous, intraarterial, intradermal, transdermal, oral, sublingual buccal, or nasal route of administration.

37. The method of claim 27, comprising administering the compound as a single dose.

38. The method of claim 27, comprising administering the compound in repeated doses.

39. The method of claim 27, wherein administration of the compound induces neurite outgrowth.

40. The method of claim 27, further comprising the administration of a therapeutic agent.

41. The method of claim 40, wherein the compound or derivative thereof is administered prior to, simultaneously with or following administration of the therapeutic agent.
